(19) United States
(12) Patent Application Publication
(10) Pub. No.: US 2023/0035945 A1
(43) Pub. Date: Feb. 2, 2023

(54) Mouthpiece For A Product Dispensing Device

(71) Applicants: Nemera La Verpillière, LA VERPILLIERE (FR); UNIVERSITÉ DE TOURS, TOURS CEDEX 1 (FR); Institut National de la Sante et de la Recherche Medical (Insem), Paris (FR)

(72) Inventors: Alain Regard, Beynost (FR); Guillaume Grevin, L'Isle d'Abeau (FR); Sébastien Delvalac, LYON (FR); Déborah Le Pennec, MONTS (FR); Laurent Vecellio, CHAMBRAY LES TOURS (FR)

(21) Appl. No.: 17/785,002

(22) PCT Filed: Dec. 11, 2020
(86) PCT No.: PCT/EP2020/085851

§ 371 Date: Jun. 13, 2022

(30) Foreign Application Priority Data
Dec. 11, 2019 (FR) FR1914200

(51) Int. Cl. A61M 15/00; A61M 16/04

(52) U.S. Cl. CPC A61M 15/0021; A61M 15/009; A61M 16/0495

(57) ABSTRACT

The invention relates to a mouthpiece for a product dispensing device including a product dispensing duct defining a longitudinal axis and including a connection opening designed to be connected to a dispensing member of the product dispensing device, and a dispensing opening designed to open freely into the mouth of a user, and a lingual cavity designed to receive at least one end of the tongue of the user, including a lingual opening allowing the tongue to be inserted into the lingual cavity and a contact surface designed to form a target for one end of the tongue of the user.

The invention also relates to a product dispensing device and to a method for dispensing a product.

### FIELD OF THE INVENTION

The invention relates to a mouthpiece for a product dispensing device.

### SUMMARY OF THE INVENTION

The state of the art already describes mouthpieces for product dispensing devices. For example, such mouthpieces for aerosol administration devices are known, these devices generally comprising a generator of particles, for example of size between 10 nanometers and 200 micrometers, a pressure source to transport the particles and a mouthpiece to administer the aerosol via the mouth. The pressure source may be endogenous, made up for example of an inhaling pressure obtained by the user breathing in through the mouth. Alternatively, the pressure source may be exogenous, made up for example of a pressurized gas reservoir.

The particles, for example of a medication, may be in liquid or solid form. In the case of particles in liquid state, the particle generator is for example in the form of an atomizer used to produce a spray. In the case of particles in solid state, the particle generator delivers for example a predetermined quantity of powder on a support, said support then being for example subjected to the action of a propulsive flow.

In some cases, the medication may be deposited in the nasal cavities after passing through the mouth. Alternatively, the medication may be deposited in the lungs, after passing through the mouth.

An aerosol administration device, as disclosed in document FR2954707, is known for example. Such a device includes a mouthpiece used to administer an aerosol through the mouth during a nasal exhalation phase or during a respiratory pause phase preceding the nasal exhalation. In this device, the mouthpiece is airtight and extends beyond the patient's teeth over a certain length. Thus, in this case, the mouthpiece forms a means for administering the aerosol for the nasal, nasopharynx or nasosinusian region. The aerosol containing the particles is thus routed successively in the mouth, the nasopharynx and then the nasal cavities and the sinuses, until it escapes via at least one of the patient's nostrils.

However, it may not be desirable to deposit product in the mouth. Such a deposit reduces the product dispensing efficiency, and therefore the dose actually received by the target organ, since the product is deposited in the mouth instead of the desired treatment zone. In addition, the product may also affect or at least disturb the sense of taste of a user.

Thus, due to their shape, the current mouthpieces do not prevent, or only partially prevent, product from being deposited in the mouth.

The invention aims in particular to propose a mouthpiece, which guarantees that as much as possible of the dose dispensed is received by the target organ.

The invention therefore relates to a mouthpiece for a product dispensing device, comprising:
a product dispensing duct defining a longitudinal axis and comprising a connection opening designed to be connected to a dispensing member of the product dispensing device, and a dispensing opening designed to open freely into the mouth of a user; and
a lingual cavity designed to receive at least one end of the tongue of the user, comprising a lingual opening allowing the tongue to be inserted into the lingual cavity and a contact surface designed to form a target for one end of the tongue of the user.

Thus, it is proposed to perform the invention by creating a cavity for the tongue of the user, which further comprises a haptic target for one end of the tongue of the user, so that the tongue of the user is incited to move forward, and therefore to lower, so that the respiratory tracts or airways behind the tongue become more open. This minimizes the quantity of product deposited in the mouth, for example on the tongue.

By reaching the target with his tongue, or at least by trying to reach it with his tongue, the user ensures that as much as possible of the dose dispensed reaches the target organ and that less product is wasted in the mouth.

According to other optional characteristics of the mouthpiece, taken alone or in combination:
the lingual cavity is at least partially adjacent to the dispensing duct. Thus, the mouthpiece takes less space in the mouth.
the mouthpiece further comprises a mouth opener surrounding at least partially the dispensing duct and the lingual cavity. Thus, due to the mouth opener, the user is forced to open the mouth wide, so that the lower jaw of the user is lowered, which makes it easier to access the back of the mouth and consequently improves the dispensing of product in zones located beyond the mouth, in other words downstream from the mouth in the direction of the product dispensing flow. In addition, the mouth opener can be used to position the mouthpiece. It is understood that the mouth opener advantageously acts as support for the dispensing duct and the lingual cavity.
the mouth opener comprises a peripheral wall preferably having the shape of a surface of revolution or of a cylinder of elliptical or ovoid cross-section, preferably having the shape of a straight cylinder of circular cross-section. Thus, when the mouth opener is O-shaped, a seal can be made between the lips of the user and the mouth opener, thus preventing product from coming out of the mouth cavity. A surface of revolution is obtained by rotating a curve or a straight line about an axis of revolution. A surface of revolution is for example a cylinder, which may for example be a straight cylinder of circular cross-section. In this case, the support is more comfortable. Alternatively, the surface of revolution may for example be conical, thereby allowing the mouthpiece to adapt to mouths of different sizes.
the mouthpiece further comprises a front support surface for the lips of the user. Thus, the mouthpiece is axially precisely positioned in the mouth of the user, simply and repeatably for the user. In addition, the sealing of the dispensing is improved.
the front support surface lies in a plane defined by the contact surface. Thus, the device is easier to clean, especially the lingual cavity, between two uses.
the mouthpiece comprises a plate having the front support surface and having a single opening delimiting the connection opening. Thus, since there is a single opening, the ambient air has no influence that could have a negative effect on the dispensing of product and consequently on the deposit of product in the mouth.
the value of an angle formed between the longitudinal axis of the dispensing duct and the front support surface is between 90 degrees and 120 degrees, preferably between 92 degrees and 100 degrees, more preferably close to 97.5 degrees. Thus, the angle formed by the dispensing duct relative to the mouth of the user makes it possible to direct product towards the back of the throat and therefore reduce the quantity of product deposited in the top of the mouth of the user. Note that the longitudinal axis of the dispensing duct can be an axis of revolution of the dispensing duct, or more generally if it is not an axis of revolution, a central axis of the dispensing duct.
the front support surface is substantially perpendicular to a central axis of the mouth opener. Thus, the mouthpiece is positioned more precisely axially in the mouth of the user.
the lingual cavity is at least partially aligned with a lower part of the dispensing duct. Thus, the lower part of the dispensing duct can be used to guide the tongue of the user comfortably towards the lingual cavity.
the dispensing duct further comprises at least one air passage opening, which is preferably arranged on the upper part of the dispensing duct opposite the lingual cavity. Thus, product is dispensed faster and more efficiently due to the Venturi effect caused during the product dispensing flow by air sucked through the air passage opening.
the length of the dispensing duct between the front support surface and the dispensing opening is between 30 millimeters and 70 millimeters, preferably between 50 millimeters and 60 millimeters, more preferably close to 55 millimeters. Thus, the length of the duct is optimum to limit the deposit of product in the mouth, while remaining comfortable for the user.
the inner wall of the dispensing duct has the shape of a surface of revolution or of a cylinder of elliptical cross-section, preferably the shape of a straight cylinder of elliptical cross-section. Thus, the product dispensing flow is improved. A surface of revolution is obtained by rotating a curve or a straight line about an axis of revolution. A surface of revolution is for example a cylinder, which may for example be a straight cylinder of circular cross-section. Alternatively, the surface of revolution may for example be conical.
the connection opening is designed to receive a dispensing member of the product dispensing device or the product dispensing device itself. Thus, the dispensing duct is particularly easy to connect to the dispensing member of the product dispensing device. In other words, the connection opening has the shape of a reception cavity, which is located upstream from the product dispensing opening, considering the direction of the product dispensing flow.
the mouthpiece comprises means for removably attaching it to a dispensing member of a product dispensing device, preferably comprising the connection opening, which preferably has a conical shape. Thus, the mouthpiece can easily be installed on an existing product dispensing device, for example already provided with a mouthpiece. Since the attachment means are removable, the mouthpiece can also be removed, for example to clean it. When the connection opening has a conical shape, the dispensing member and the mouthpiece are removably attached simply and sealably by tightening the dispensing member into the connection opening.
the lingual cavity comprises means for detecting the position of the tongue, preferably a proximity sensor or a contact sensor on the contact surface. Thus, information relating to the position of the tongue of the user can be sent to the user or to the product dispensing device, for example the user or the product dispensing device can then trigger the dispensing of product, if the tongue of the user is detected to be in the correct position.
the lingual cavity comprises indication means for indicating that the tongue of the user is in the correct position, preferably indication means comprising a textured surface arranged on the contact surface. Thus, the information relating to the correct position of the tongue of the user is particularly easy to obtain. A textured surface means in particular a surface with sufficient roughness or reliefs to allow tactile detection by the tongue of the user.

The invention also relates to a product dispensing device, comprising:
a dispensing member, and
a mouthpiece of the above-mentioned type.

The invention further relates to a method for dispensing a product using a product dispensing device of the above-mentioned type, comprising at least the following steps:
inserting the mouthpiece into the mouth of a user,
inserting the tongue of the user into the lingual cavity, and
triggering the dispensing of product when the tongue of the user touches the contact surface.

### BRIEF DESCRIPTION OF THE DRAWINGS

The invention will be better understood on reading the following description, given solely by way of example and with reference to the accompanying drawings in which:

FIG. 1 is a rear perspective view of a mouthpiece according to a first embodiment;

FIG. 2 is a front perspective view of the mouthpiece according to the first embodiment shown on FIG. 1;

FIG. 3 is a cross-sectional view of the mouthpiece according to the first embodiment shown on FIG. 1;

FIG. 4 is a rear perspective view of a product dispensing device comprising a mouthpiece according to the first embodiment;

FIG. 5 is a cross-sectional view of the product dispensing device comprising a mouthpiece according to the first embodiment and a dispensing member according to a first variant;

FIG. 6 is a cross-sectional view of a second product dispensing device comprising a mouthpiece according to the first embodiment and a dispensing member according to a second variant;

FIG. 7 is a rear perspective view of a mouthpiece according to a second embodiment;

FIG. 8 is a rear perspective view of a mouthpiece according to a third embodiment; and

FIG. 9 is a cross-sectional view of the second product dispensing device shown on FIG. 6, in which the mouthpiece is inserted into the mouth of a user.

### DETAILED DESCRIPTION OF THE INVENTION

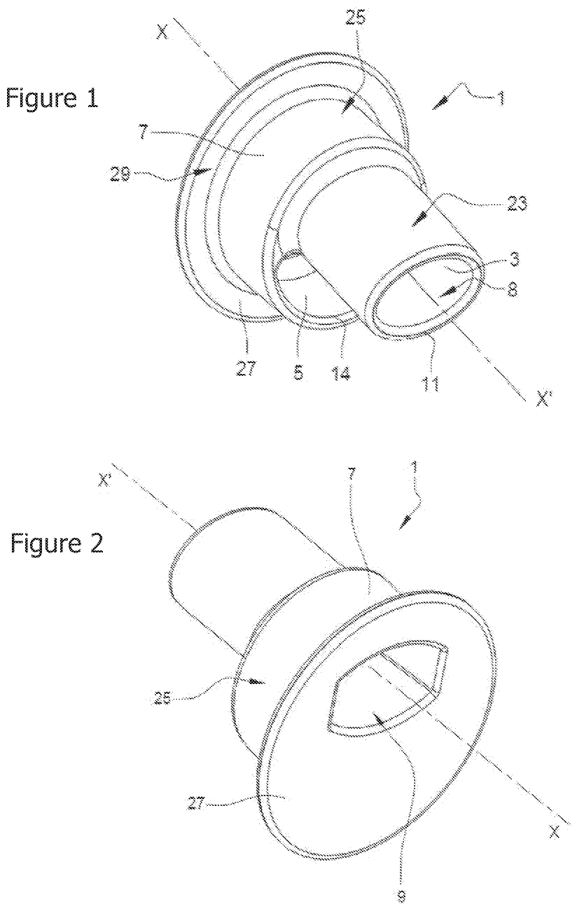

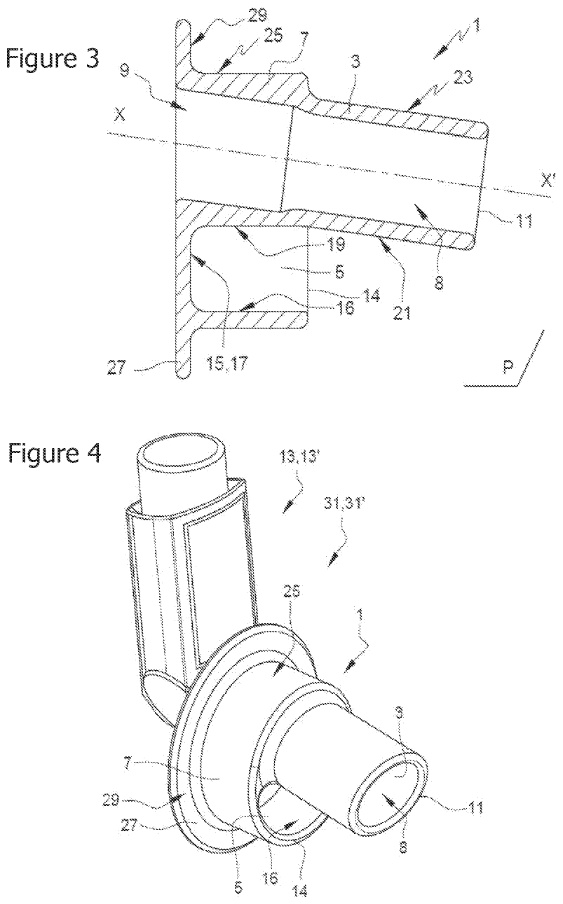

FIGS. 1 to 3 show a mouthpiece according to a first embodiment of the invention, designated by the general reference 1. The mouthpiece 1 is substantially symmetrical relative to a plane of symmetry P, as shown on FIG. 3.

This mouthpiece 1 comprises a dispensing duct 3 and a lingual cavity 5. It further comprises a mouth opener 7.

The dispensing duct 3 is designed to dispense a product, for example an aerosol. The dispensing duct 3 extends longitudinally, thus defining a longitudinal axis XX′. In this first embodiment, the dispensing duct 3 has the shape of a hollow cylinder of elliptical cross-section. Thus, the inner wall 8 of the dispensing duct 3 has the shape of a straight cylinder of elliptical cross-section.

The dispensing duct 3 comprises a connection opening 9 and a dispensing opening 11.

The connection opening 9 is designed to be connected to a dispensing member 13, 13′, as shown on FIGS. 4 to 6. The purpose of the dispensing member 13, 13′ is to dispense a product, for example when activated by a user.

The dispensing opening 11 is designed to open freely into the mouth of a user. Thus, the dispensing opening 11 forms the downstream end of the dispensing duct 3, in the direction of the product flow when dispensing product.

The lingual cavity 5 is designed to receive at least one end of the tongue of the user. The lingual cavity 5 comprises a lingual opening 14 allowing the tongue of the user to be inserted into the lingual cavity 5. The lingual cavity 5 also comprises a contact surface 15 designed to form a target for one end of the tongue of the user. In this first embodiment, the contact surface 15 is at the back of the lingual cavity 5. As an alternative or in addition, the contact surface can be arranged on the side surface 16 of the lingual cavity 5. For example, the lingual cavity 5 is 20 millimeters deep.

The lingual cavity 5 further comprises indication means for indicating that the tongue of the user is in the correct position. In the example shown on FIGS. 1 to 3, the indication means comprise a textured surface 17 arranged on the contact surface 15. As an alternative or in addition, the lingual cavity 5 may comprise means for detecting the position of the tongue, for example a proximity sensor or a contact sensor on the contact surface 15.

The lingual cavity 5 is at least partially adjacent to the dispensing duct 3. In addition, the lingual cavity 5 is at least partially aligned with a lower part of the dispensing duct 3. More precisely, as shown on FIG. 3, the upper part 19 of the side surface 16 of the lingual cavity 5 is connected to the lower part 21 of the peripheral surface 23 of the dispensing duct 3.

As shown in particular on FIGS. 1 to 3, the mouth opener 7 comprises a peripheral wall 25 having the shape of a straight cylinder of circular cross-section. For example, the diameter of the peripheral wall 25 is 40 millimeters.

As shown in particular on FIG. 3, the mouth opener 7 surrounds at least partially the dispensing duct 3 and the lingual cavity 5. More precisely, the lingual cavity 5 is arranged in the mouth opener 7, and the dispensing duct 3 projects partially from the mouth opener 7, on the side of the lingual opening 14 of the lingual cavity 5. For example, the outer dimensions of the elliptical cross-section of the peripheral part 23 of the dispensing duct 3 projecting from the mouth opener 7 are as follows: large axis of length 29 millimeters, small axis of length 20 millimeters. For example, the length of the peripheral part 23 of the dispensing duct 3 projecting from the mouth opener 7 is about 30 millimeters, in particular 28.7 millimeters in the lower part 21 of the peripheral surface 23 in the plane of symmetry P. For example, the thickness of the peripheral part 23 of the dispensing duct 3 is from 0.8 millimeters to 2.5 millimeters, preferably close to 1.3 millimeters. The lingual cavity 5 has a contour which conforms to the shape of the mouth opener 7 and of the dispensing duct 3, and thus has substantially the shape of a portion of a ring, as shown on FIG. 1, whose maximum vertical height is for example 13.4 millimeters in the plane of symmetry P.

The mouthpiece 1 also comprises a plate 27 which comprises a front support surface 29 for the lips of the user.

The plate 27 is arranged on one end of the mouth opener 7, on the side opposite the lingual opening 14 of the lingual cavity 5. The front support surface 29 is thus perpendicular to a central axis of the mouth opener 7. The plate 27 further comprises a single opening which delimits the connection opening 9. For example, the plate 27 is 2.5 millimeters thick.

In the example shown, the front support surface 29 lies in a plane defined by the contact surface 15. Thus, the plate 27 also carries the contact surface 15 located at the back of the lingual cavity 5.

The value of an angle formed between the longitudinal axis XX′ of the dispensing duct 3 and the front support surface 29 is between 90 degrees and 120 degrees, preferably between 92 degrees and 100 degrees. According to an example not shown, the value of the angle is 92.5 degrees. According to another example not shown, the value of the angle is 95 degrees. In the example shown on the figures, the value of this angle is 97.5 degrees.

As shown on FIGS. 4 to 6, the mouthpiece 1 may form an element of a product dispensing device 31, 31′. Thus, the product dispensing device 31, 31′ comprises a dispensing member 13, 13′ and a mouthpiece 1. The mouthpiece 1 can therefore be reused.

The dispensing member 13, 13′ comprises a product reservoir 33 and dispensing means 35, which comprise for example a valve or a pump. When the dispensing member 13, 13′ is activated, the dispensing means 35 distribute product towards an outlet opening 37 of the dispensing member 13, 13′. As shown on FIGS. 4 to 6, the connection opening 9 consists of a cavity having a shape complementary to that of a dispensing member 13,13′ of the product dispensing device 31, 31′. More precisely, the connection opening 9 has a shape complementary to that of the peripheral wall 38 delimiting the outlet opening 37 of the dispensing member 13, 13′ of the product dispensing device 31, 31′. Thus, the connection opening 9 has a shape complementary to that of the outlet opening 37. The mouthpiece 1 therefore comprises means for removably attaching it to the dispensing member 13, 13′ of the product dispensing device 31, 31′. For example, the connection opening 9 preferably has a cross-section having the shape of a deformed rectangle, whose lengths are formed by circular arcs. In addition, the connection opening 9 may have a conical shape.

To obtain a sealed connection by tightening the dispensing member 13, 13′ into the connection opening 9 of the mouthpiece 1, the connection opening 9 is for example about 20 millimeters deep.

The length of the dispensing duct 3 between the front support surface and the dispensing opening 11 is between 30 millimeters and 70 millimeters, preferably between 50 millimeters and 60 millimeters, in the example shown close to 55 millimeters.

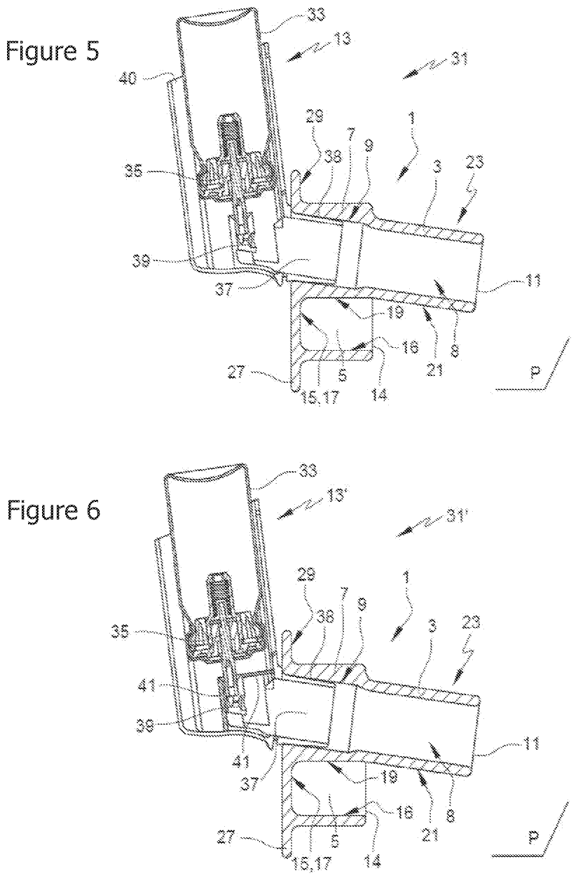

For example, as shown on FIGS. 5 and 6, the maximum longitudinal distance between a dispensing nozzle 39 of the dispensing means 35 and the dispensing opening 11 is 90 millimeters, for example 65 millimeters.

In a first variant shown on FIG. 5, the dispensing member 13 is not sealed. When dispensing is triggered, in fact, the outside air, i.e. outside the user, passes from an inlet opening 40 of the dispensing member 13 towards the outlet opening 37.

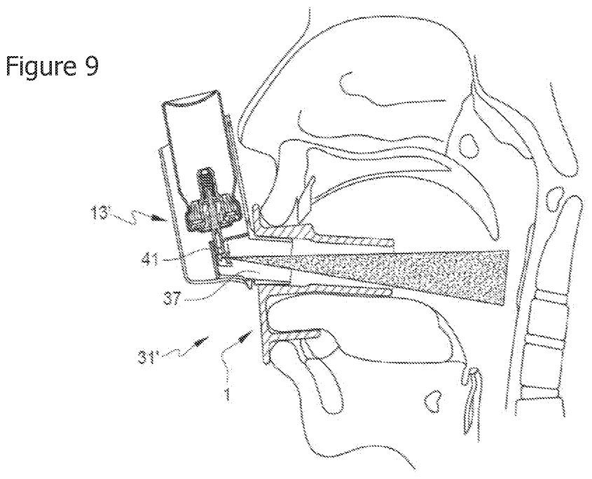

In a second variant shown on FIG. 6, the dispensing member 13′ comprises an inner wall 41 which prevents any outside air from passing towards the outlet opening 37. Thus, the dispensing member 13′ can be used to dispense product without outside air entering the mouth of the user via the dispensing device 31′. FIG. 9 shows a product dispensing device 31′ comprising such a dispensing member 13′, whose mouthpiece 1 is inserted into the mouth of a user.

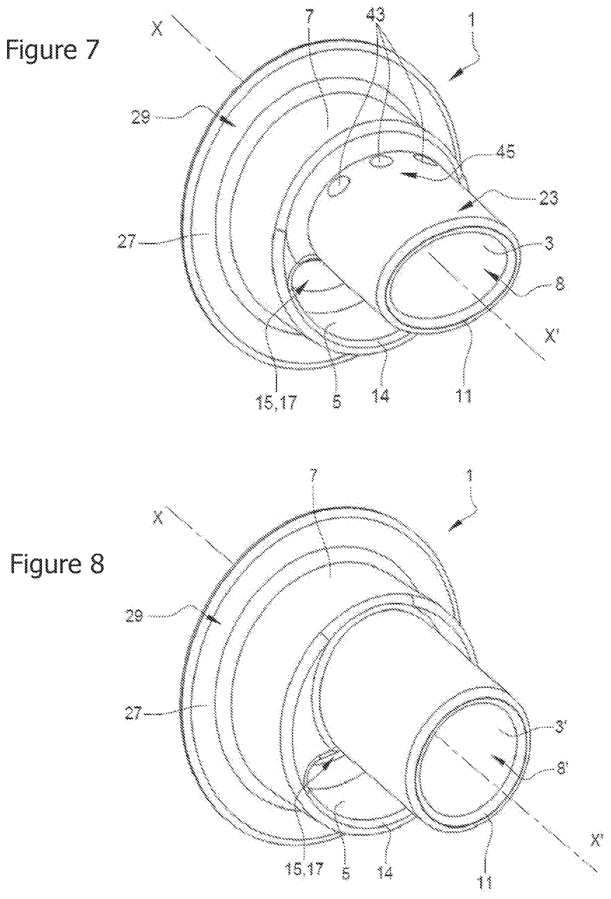

A second embodiment shown on FIG. 7 is similar to the embodiment previously described, apart from the fact that the dispensing duct 3 comprises air passage openings 43. In the example shown, the dispensing duct 3 comprises three air passage openings 43, which are uniformly distributed over the upper part 45 of the peripheral surface 23 of the dispensing duct 3. As an alternative or in addition, air passage openings could be arranged on one or the lateral part(s) of the peripheral surface 23 of the dispensing duct 3. The upper part 45 is opposite the lingual cavity 5, in other words opposite the lower part 21 of the peripheral surface 23 of the dispensing duct 3. In the example shown on FIG. 7, the axes of the air passage openings 43 lie in the same plane, which is orthogonal to the plane of symmetry P of the mouthpiece 1. The air passage openings 43 are arranged near the mouth opener 7, so that when the user closes their lips on the mouth opener 7, the air inside the mouth of the user can pass through the openings 43. Since they are arranged near the mouth opener 7, the air passage openings 43 have an effect which favors the flow when product is dispensed in the dispensing duct 3. The openings 43 produce a Venturi effect after injecting product through the opening 39. The air in the mouth cavity enters through the openings 43 to favor efficient transport of the medication particles through the dispensing duct 3, thereby limiting the loss of medication in the duct 3. Such a configuration with air passage openings 43 is particularly advantageous in an airtight product dispensing device 31′, in which the dispensing member 13′ comprises an inner wall 41 which prevents any outside air from passing towards the outlet opening 37, as shown previously on FIG. 6.

A third embodiment shown on FIG. 8 is similar to the first embodiment previously described, apart from the fact that the dispensing duct 3′ has the shape of a hollow cylinder of circular cross-section. Thus, the inner wall 8′ of the dispensing duct 3′ has the shape of a straight cylinder of circular cross-section, of diameter between 20 and 30 millimeters, in this example 20 millimeters.

In the embodiments previously described, the mouthpiece 1 is made of at least one polymer material. The mouthpiece 1 can be manufactured using an injection molding process.

A method for dispensing a product using a product dispensing device 31, 31′ as previously described is also described.

Such a dispensing method comprises at least the following steps:
inserting the mouthpiece 1 into the mouth of a user,
inserting the tongue of the user into the lingual cavity 5, and
triggering the dispensing of product when the tongue of the user touches the contact surface 15.

Note that this last step of triggering the dispensing can be performed manually by the user. Thus, when the user detects that their tongue touches the contact surface 15, they can trigger the dispensing of product, for example by detection by means of the textured surface 17.

Alternatively, when the tongue of the user touches the contact surface 15, the product dispensing device 31, 31′ can automatically trigger the dispensing of product via the product dispensing means 35 of the dispensing member 13, 13′. The tongue of the user is detected as touching the contact surface 15, for example, by means for detecting the position of the tongue, for example a proximity sensor or a contact sensor on the contact surface 15.

The invention is not limited to the embodiments described and other embodiments will be clearly apparent to those skilled in the art. In particular, the second and third embodiments can be combined together. The example described in the first embodiment can be applied by those skilled in the art to the second and third embodiments. The dimensions indicated are those corresponding to use by an adult user. Those skilled in the art will know how to adapt these dimensions to adapt them for pediatric use.

### LIST OF REFERENCES

1: mouthpiece
3, 3′: dispensing duct
5: lingual cavity
7: mouth opener
8, 8′: inner wall (of the dispensing duct 3, 3′)
9: connection opening
11: dispensing opening
13, 13′: dispensing member
14: lingual opening
15: contact surface
16: side surface
17: back surface
19: upper part (of the side surface 16)
21: lower part (of the peripheral surface 23)
23: peripheral surface (of the dispensing duct 3)
25: peripheral wall (of the mouth opener 7)
27: plate
29: front support surface
31, 31′: product dispensing device
33: product reservoir
35: dispensing means
37: outlet opening (of the dispensing member 13, 13′)
38: peripheral wall
39: dispensing nozzle
40: inlet opening
41: inner wall
43: air passage opening
45: upper part (of the peripheral surface 23)
P: plane of symmetry

1. A mouthpiece for a product dispensing device comprising:
a product dispensing duct defining a longitudinal axis and comprising a connection opening configured to be connected to a dispensing member of the product dispensing device, and a dispensing opening designed to open freely a mouth of a user, and
a lingual cavity configured to receive at least one end of a tongue of the user, the lingual cavity comprising a lingual opening allowing the tongue to be inserted into the lingual cavity and a contact surface designed to form a target for one end of the tongue of the user.

2. The mouthpiece according to claim 1, further comprising a mouth opener at least partially surrounding the dispensing duct (3, 3′) and the lingual cavity.

3. The mouthpiece according to claim 1, further comprising a front support surface for lips of the user.

4. The mouthpiece according to claim 2, comprising a plate having the front support surface and having a single opening delimiting the connection opening.

5. The mouthpiece according to claim 3, wherein a value of an angle formed between the longitudinal axis of the dispensing duct and the front support surface is between 90 degrees and 120 degrees, preferably between 92 degrees and 100 degrees, and more preferably close to 97.5 degrees.

6. The mouthpiece according to claim 1, wherein the dispensing duct further comprises at least one air passage opening arranged on an upper part of the dispensing duct opposite the lingual cavity.

7. The mouthpiece according to claim 1, wherein the lingual cavity comprises a detection member for detecting the position of the tongue, the detection member comprising a proximity sensor or a contact sensor on the contact surface.

8. The mouthpiece according to claim 1, wherein the lingual cavity comprises an indication member for indicating that the tongue of the user is in a correct position, the indication member comprising a textured surface arranged on the contact surface.

9. A product dispensing device comprising:
a dispensing member, and
the mouthpiece according to claim 1.

10. A method for dispensing a product using the product dispensing device according to claim 9, comprising at least the following steps:
inserting the mouthpiece into the mouth of the user,
inserting the tongue of the user into the lingual cavity, and
triggering the dispensing of the product when the tongue of the user touches the contact surface.
